(19) United States
(12) Patent Application Publication
(10) Pub. No.: US 2021/0121250 A1
(43) Pub. Date: Apr. 29, 2021

(54) ENDOSCOPE SYSTEM, METHOD FOR CALIBRATING ENDOSCOPE, AND ENDOSCOPE CONTROL DEVICE

(71) Applicant: OLYMPUS CORPORATION, Tokyo (JP)

(72) Inventor: Kensuke UESUGI, Tokyo (JP)

(73) Assignee: OLYMPUS CORPORATION, Tokyo (JP)

(21) Appl. No.: 17/142,516

(22) Filed: Jan. 6, 2021

Related U.S. Application Data
(63) Continuation of application No. PCT/JP2018/026181, filed on Jul. 11, 2018.

(51) Int. Cl. A61B 34/30; A61B 1/05; A61B 1/005; A61B 1/00

(52) U.S. Cl. CPC A61B 34/30; A61B 1/05; A61B 1/0052; A61B 2034/301; A61B 1/00006; A61B 2560/0223; A61B 1/0016

(57) ABSTRACT

Provided is an endoscope system including: an elongated section having a longitudinal axis; a bending section connected to a distal end of the elongated section, a tilt angle of the bending section relative to the longitudinal axis being changed; an image-capturing unit provided at a distal end of the bending section; a first motor that drives the bending section; a second motor that rotates the elongated section about the longitudinal axis; and a controller that processes an image captured by the image-capturing unit, wherein the controller acquires, from the image-capturing unit, a plurality of images that are captured while the elongated section is being rotated by the second motor, and calculates a driving direction of the bending section for making a center position of the image coincide with a position of a fixed point of the acquired plurality of images.

### CROSS-REFERENCE TO RELATED APPLICATIONS

This is a continuation of International Application PCT/JP2018/026181, with an international filing date of Jul. 11, 2018, which is hereby incorporated by reference herein in its entirety.

### TECHNICAL FIELD

The present invention relates to an endoscope system, a method for calibrating an endoscope, and an endoscope control device.

### BACKGROUND ART

There are well-known endoscopes that perform a so-called centering movement in which a bending section provided at the distal end of an insertion section is automatically moved into a linearly extended state (refer to, for example, PTL 1).

In the endoscope in PTL 1, the amount of rotation of each motor for moving the bending section is detected by a potentiometer, a residual bending angle is estimated on the basis of the detected amount of rotation, and the motor is operated by the estimated residual bending angle.

### CITATION LIST

### Patent Literature

### {PTL 1}

Patent Application, Publication No. 2002-323661

### SUMMARY OF INVENTION

One aspect of the present invention is an endoscope system including: an elongated section having a longitudinal axis; a bending section connected to a distal end of the elongated section, a tilt angle of the bending section relative to the longitudinal axis being changed; an image-capturing unit provided at a distal end of the bending section; a first motor that drives the bending section; a second motor that rotates the elongated section about the longitudinal axis; and a controller that processes an image captured by the image-capturing unit, wherein the controller acquires, from the image-capturing unit, a plurality of images that are captured while the elongated section is being rotated by the second motor, and calculates a driving direction of the bending section for making a center position of the image coincide with a position of a fixed point of the acquired plurality of images.

In addition, another aspect of the present invention is a method for calibrating an endoscope that includes an image-capturing unit provided at a distal end of a benging section and an elongated section, the method including: acquiring a plurality of images that are captured by the image-capturing unit while the elongated section is being rotated; calculating a driving direction of the bending section for making a center position of an image coincide with a position of a fixed point of the acquired plurality of images; and moving the bending section on the basis of the calculated driving direction.

In addition, another aspect of the present invention is a control device of an endoscope, the control device including: at least one processor, wherein the processor is configured to: acquire, from an image-capturing unit of the endoscope, a plurality of images that are captured while an elongated section of the endoscope is being rotated about a longitudinal axis of the elongated section; calculate a driving direction of a bending section of the endoscope for making a center position of an image coincide with a position of a fixed point of the acquired plurality of images.

### BRIEF DESCRIPTION OF DRAWINGS

FIG. 1 is an overall configuration diagram showing an endoscope system according to one embodiment of the present invention.

FIG. 2 is a diagram showing an insertion section of an endoscope included in the endoscope system in FIG. 1 and an example of feature points in a subject.

FIG. 3 is a diagram showing an example of an image that is captured by an image-capturing unit of the endoscope in FIG. 2.

FIG. 4 is a diagram showing an example of an image that is captured when the insertion section is rotated by 90° about a longitudinal axis from the state in FIG. 2.

FIG. 5 is a diagram showing an example of an image that is captured when the insertion section is rotated by 180° about the longitudinal axis from the state in FIG. 2.

FIG. 6 is a diagram showing an example of an image that is captured when the insertion section is rotated by 270° about the longitudinal axis from the state in FIG. 2.

FIG. 7 is a diagram showing an example of motion vectors for each of the feature points calculated from the images in FIGS. 3 to 6.

FIG. 8 is a flowchart for illustrating a method for calibrating the endoscope by using the endoscope system in FIG. 1.

FIG. 9 is a flowchart for illustrating the fixed-point detection step in FIG. 8.

FIG. 10 is a flowchart for illustrating the bending-section drive step in FIG. 8.

FIG. 11 is a flowchart for illustrating a modification of the bending-section drive step in FIG. 10.

FIG. 12 is a diagram showing an example of an endoscope including a control device.

FIG. 13 is an overall configuration diagram showing a modification of the endoscope system in FIG. 1.

### DESCRIPTION OF EMBODIMENTS

An endoscope system 1, a method for calibrating an endoscope 2, and a control device 10 of the endoscope 2 according to one embodiment of the present invention will now be described with reference to the drawings.

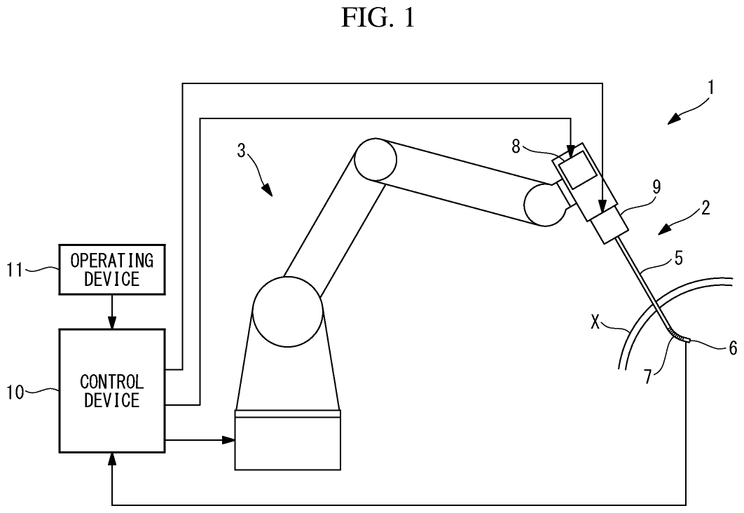

As shown in FIG. 1, the endoscope system 1 according to this embodiment includes: the endoscope 2 that is inserted into a body cavity X of a patient and that captures an image (refer to FIG. 3) G of the interior of the body cavity X; a robot 3 that can adjust the position and orientation of the endoscope 2; and the control device 10 for processing the image G captured by the endoscope 2.

The endoscope 2 includes: an elongated insertion section (elongated section) 5 that is inserted via a hole formed in a body wall of the patient; an image-capturing unit 6 provided at the distal end of the insertion section 5; a bending section 7 for changing the tilt angle of the field of view obtained with the image-capturing unit 6 relative to a longitudinal axis K of the insertion section 5; a bending motor (bending drive unit) 8 for driving the bending section 7; and a roll motor (rotation drive unit) 9 for rotating the insertion section 5 about the longitudinal axis K.

The robot 3 may be a general-purpose six-axis multi-joint robot for supporting the endoscope 2 at the distal end of a wrist.

The robot 3 and the endoscope 2 are connected to the control device 10, and an operating device 11 is connected to the control device 10.

The operating device 11 is a device that is operated when an operator remotely operates the endoscope 2 and the robot 3 and allows the operator to input a calibration start command.

When a calibration start command is input from the operating device 11, the control device 10 actuates the endoscope 2 by actuating the roll motor 9 of the endoscope 2. Thereafter, while the insertion section 5 is being rotated about the longitudinal axis K, the control device 10 acquires a plurality of images G. The control device 10 is configured from a computer including a processor and a memory.

When the plurality of images G captured while the insertion section 5 is being rotated about the longitudinal axis K are input thereto, the control device 10 processes the plurality of input images G and calculates the position of a fixed point in an image G, thereafter actuating the bending motor 8 on the basis of information for bringing the calculated position of the fixed point closer to a center position (center) P in the image G.

Note that, for the plurality of images G, it is sufficient if a plurality of images G that differ from one another at least in the rotation angle position are acquired. More specifically, a plurality of images G are acquired in accordance with the frame rate of the image-capturing unit 6 while the insertion section 5 is being rotated (images G are acquired time-sequentially). Alternatively, an arbitrary time interval or a random time interval may be preset by providing the control device 10 with a timer function, so that a plurality of images G may be acquired at those time intervals. Alternatively, the rotation angle of the insertion section 5 may be acquired from an encoder (not shown in the figure) of the roll motor 9, so that a plurality of images G may be acquired at predetermined rotation angles on the basis of the detected value of the encoder.

Here, calculation of the position of a fixed point will be described below.

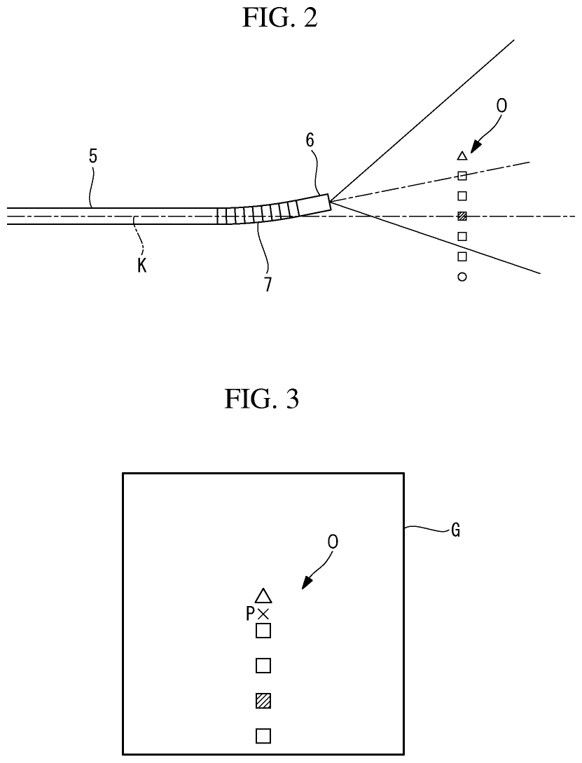

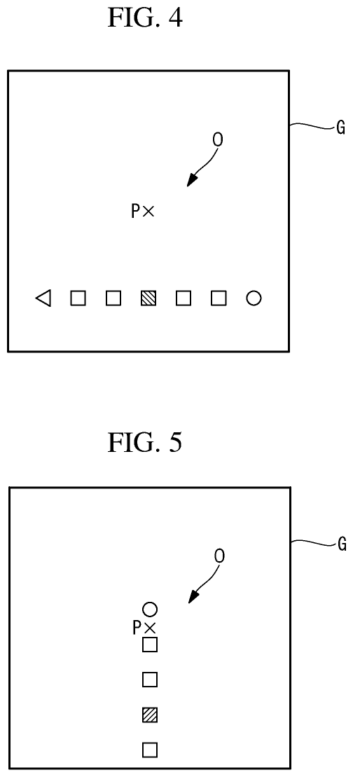

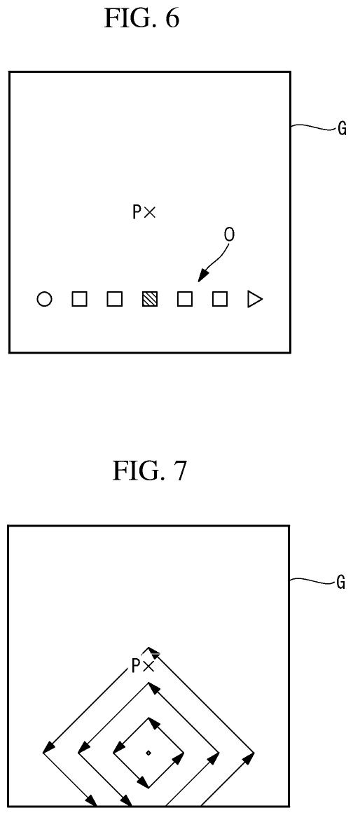

In the case where a plurality of feature points are present on a subject O as shown in FIG. 2, the image G as shown in FIG. 3 is captured by the image-capturing unit 6 if the bending section 7 is slightly bent in one direction in the initial state. When an image G is captured by the image-capturing unit 6 every predetermined angle, for example, every 90° while the endoscope 2 is rotated by one rotation from the above-described initial state about the longitudinal axis K of the insertion section 5 by actuating the roll motor 9, a total of four images G are captured, as shown in FIGS. 3 to 6.

When these images G are input thereto, the control device 10 calculates motion vectors of each of the feature points. As shown in FIG. 7, even though the insertion section 5 is rotated with the bending section 7 being bent, the feature point disposed on an extension of the longitudinal axis K of the insertion section 5 is at the same position in the images G. Therefore, the feature point disposed on an extension of the longitudinal axis K has the smallest magnitudes of motion vectors among all the feature points. The control device 10 calculates the motion vector of each of the feature points between every two images G neighboring in the time axis direction and sums the magnitudes of the calculated motion vectors. By doing so, it is possible to detect, as a fixed point, the feature point having the smallest sum of the magnitudes of the calculated motion vectors.

When the coordinates of the position of the fixed point are calculated, the control device 10 calculates, as information for bringing the calculated position of the fixed point closer to the center position P of an image G, the amount of driving of the bending motor 8 required to shift the fixed point to the center position P of the image G. The control device 10 moves the bending section 7 by driving the bending motor 8 on the basis of the amount of driving. By doing so, it is possible to place the bending section 7 close to a state in which the bending section 7 extends linearly along the longitudinal axis K of the insertion section 5.

Next, a method for calibrating the endoscope 2 in the endoscope system 1 according to this embodiment will be described.

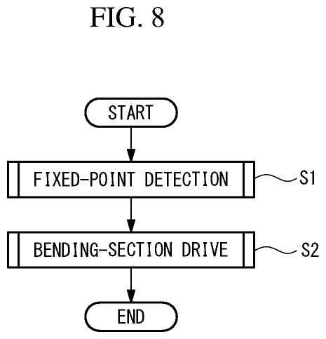

The calibration method according to this embodiment is a method for placing the bending section 7 into a state in which the bending section 7 is linearly extended along the longitudinal axis K of the insertion section 5 and, as shown in FIG. 8, includes: a fixed-point detection step S1 of calculating the position of a fixed point in an image G; and a bending-section drive step S2 of bending the bending section 7 on the basis of the calculated position of the fixed point.

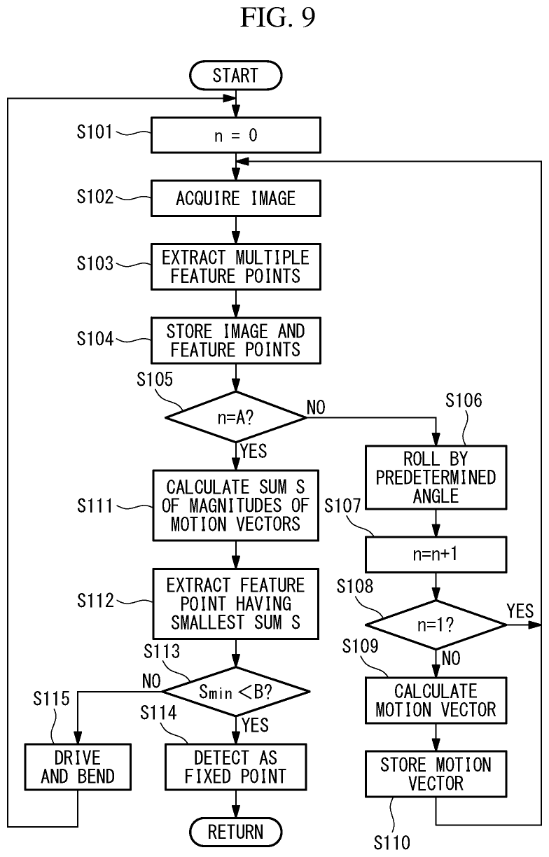

As shown in FIG. 9, in fixed-point detection step S1, when a calibration start command is input on the operating device 11, first a counter n is set to n=0 (step S101), and then an image G is captured by the image-capturing unit 6 (step S102).

The captured image G is sent to the control device 10, a plurality of feature points are extracted (step S103), and the sent image G and the coordinates of the extracted feature points are stored (step S104).

It is determined whether or not the counter indicates a predetermined number of times A (step S105). In the case where the counter does not indicate the predetermined number of times A, the roll motor 9 is actuated to rotate the insertion section 5 by a predetermined angle θ about the longitudinal axis K (step S106). Here, for example, θ=90° and A=360°/θ=4. It is acceptable if the predetermined angle θ is smaller than 180°.

The counter is incremented (step S107), and it is determined whether or not the counter n is n=1 (step S108). In the case where n=1, the process steps from step S102 are repeated. In the case where the counter n is not n=1, the motion vector of each of the feature points is calculated on the basis of the previous two images G (step S109) and is stored (step S110), and then the process steps from step S102 are repeated.

In the case where n=A is in step S105, the sum S of the magnitudes of the motion vectors that have been calculated so far is calculated for each of the feature points (step S111). Then, the sums S of the magnitudes of the calculated motion vectors are compared among the feature points, and the feature point having the smallest sum Smin is extracted (step S112). Then, it is determined whether or not the calculated smallest sum Smin is smaller than a predetermined threshold value B (step S113). In the case where the smallest sum Smin is smaller than the predetermined threshold value B, the feature point having the smallest sum Smin is detected as a fixed point, and the coordinates thereof are stored (step S114). In the case where the smallest sum Smin is equal to or larger than the predetermined threshold value B, it is possible that there are no fixed points in the images G, and hence the bending section 7 is moved (step S115), and then the process steps from step S101 are repeated.

The bending movement in step S115 can be performed by the following two methods.

The first method is performed as follows. Because it is estimated that a fixed point is present on an extended line of the straight line connecting the center position P of an image G and the feature point, extracted in step S112, having the smallest sum Smin of the magnitudes of motion vectors, the bending section 7 is bent in a direction in which the distal end thereof moves from the center position P of the image G towards the feature point having the smallest sum Smin of the magnitudes of motion vectors, whereby the fixed point, not appearing in the image G, is made to appear in the image G.

The second method is performed as follows. A search is made for a fixed point while the bending section 7 is bent by a predetermined angle at a time in an arbitrary direction without estimating the direction of the fixed point. If no fixed points are found even after several trials of bending the bending section 7 in that direction, the same processing is repeated in a different direction.

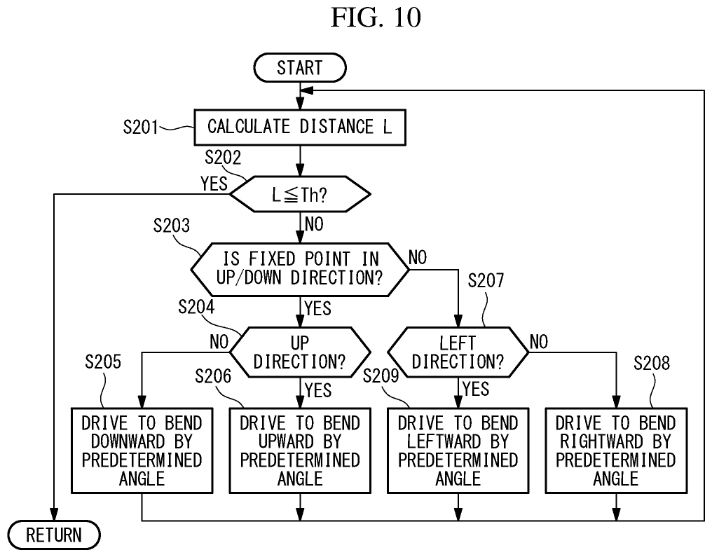

As shown in FIG. 10, in bending-section drive step S2, first the distance L from the center position P of an image G to the fixed point is calculated on the basis of the coordinates of the calculated position of the fixed point and the coordinates of the center position P of the image G (step S201). It is determined whether or not the calculated distance L is smaller than or equal to a predetermined threshold value Th (step S202). In the case where the calculated distance L is smaller than or equal to the threshold value Th, processing ends. In the case where the calculated distance L is larger than the threshold value Th, the direction of the fixed point relative to the center position P of the image G is determined (step S203).

In step S203, it is determined whether or not the fixed point is present in the up/down direction relative to the center position P of the image G. In the case where the fixed point is present in the up/down direction, it is determined whether or not the fixed point is present in the up direction (step S204). In the case where the fixed point is not present in the up direction, it can be determined that the bending section 7 is bent upward relative to the longitudinal axis K of the insertion section 5, and hence, the control device 10 moves the bending section 7 downward by a predetermined angle by controlling the bending motor 8 (step S205). On the other hand, in the case where the fixed point is present in the up direction relative to the center position P of the image G, it can be determined that the bending section 7 is bent downward relative to the longitudinal axis K of the insertion section 5, and hence, the control device 10 moves the bending section 7 upward by a predetermined angle (step S206).

In addition, in the case where it is determined that the fixed point is not present in the up/down direction in step S203, it is determined whether or not the fixed point is present in the left direction (step S207). In the case where the fixed point is not present in the left direction, it can be determined that the bending section 7 is bent leftward relative to the longitudinal axis K of the insertion section 5, and hence, the control device 10 moves the bending section 7 rightward by a predetermined angle (step S208). On the other hand, in the case where the fixed point is present in the left direction relative to the center position P of the image G, it can be determined that the bending section 7 is bent rightward relative to the longitudinal axis K of the insertion section 5, and hence, the control device 10 moves the bending section 7 leftward by a predetermined angle (step S209).

After steps S205, S206, S208, and S209 end, the process steps from step S201 are repeated. Then, in the case where it is determined that the distance L is smaller than or equal to the threshold value Th in step S202, the calibration movement ends.

According to the endoscope system 1, the method for calibrating the endoscope 2, and the control device 10 of the endoscope 2 of this embodiment, the insertion section 5 of the endoscope 2 is inserted into the body, starting with the image-capturing unit 6 provided at the distal end thereof, the insertion section 5 is rotated about the longitudinal axis K by actuating the roll motor 9, and a plurality of images G are captured by actuating the image-capturing unit 6 while the insertion section 5 is being rotated.

Then, as a result of the plurality of captured images G being processed in the control device 10, the position of a fixed point in an image G is calculated, whereby it is possible to obtain information for bringing the position of the fixed point closer to the center position P of the image G.

In other words, because rotation performed by the roll motor 9 causes the insertion section 5 to rotate about the longitudinal axis K, the images G that are captured time-sequentially by the image-capturing unit 6 during the rotation are images about the fixed point, which is a point disposed on an extended line of the longitudinal axis K. Therefore, the control device 10 drives the bending section 7 by actuating the bending motor 8 on the basis of information, thus bringing the position of the fixed point closer to the center position P of the image G. In other words, the direction of the position of the fixed point relative to the center position P of the image G can be determined, whereby it is possible to straighten out the bending section 7 with high accuracy.

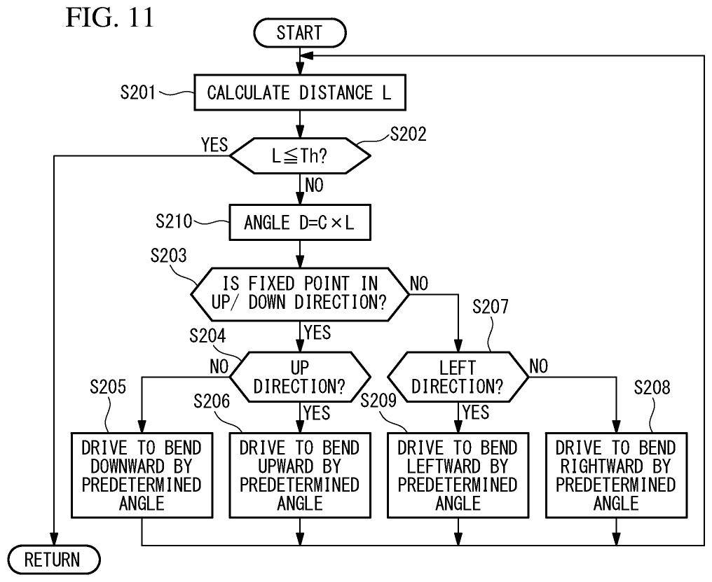

Note that although this embodiment has been described by way of an example where the bending section 7 is driven in the up, down, left, or right direction by a predetermined angle in bending-section drive step S2, instead of this, the drive angle of the bending motor 8 may be calculated on the basis of the distance L calculated in step S201, as shown in FIG. 11 (step S210). For example, a drive angle D of the bending motor 8 can be calculated by multiplying the distance L by a constant C. This provides an advantage in that, as the fixed point approaches the center position P of the image G, the drive angle D of the bending section 7 becomes smaller, whereby it is possible to straighten out the bending section 7 with higher accuracy.

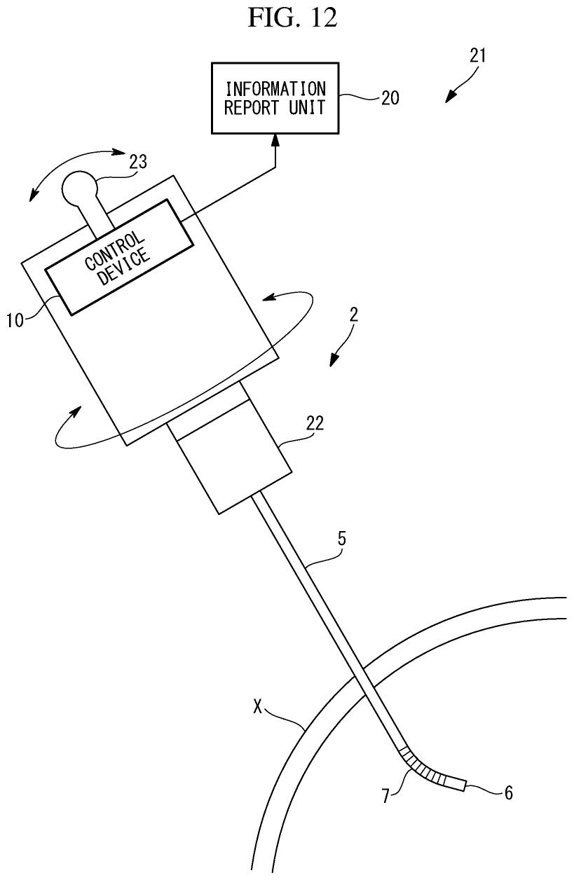

In addition, although this embodiment has been described by way of example of the endoscope system 1 in which the control device 10 controls the robot 3 and the endoscope 2 according to an operation performed with the operating device 11, the present invention is not limited to this. A manually operated endoscope 2 may be supported by a support device, such as a surgical arm, for holding the position and the orientation of the endoscope 2. It is sufficient if the endoscope 2 includes a manually operated handle (rotation drive unit: refer to FIG. 12) 22 for rotating the insertion section 5 about the longitudinal axis K and a manually operated handle (bending drive unit: refer to FIG. 12) 23 for bending the bending section 7.

In this case, it is sufficient if the endoscope system 1 includes an information report unit (refer to FIG. 12) 20 that reports information, which is output from the control device 10, for bringing the position of the fixed point closer to the center position P of the image G.

The information report unit 20 is any kind of reporting means including a display, a speaker, etc.

In the case where information is to be reported on a display, a message such as “Bend to the right by 5°” may be displayed as the information for bringing the position of the fixed point closer to the center position P of the image G. As a result of the technician actuating the endoscope 2 according to the report content in the information report unit 20, the control device 10 processes the acquired images G, whereby it is possible to bring the position of the fixed point closer to the center position P of an image G. In addition, only a bending direction may be reported without explicitly indicating an angle.

In addition, instead of preparing the robot 3 and a surgical arm, the present invention may be applied to a manually operated endoscope system 21 in which the endoscope 2 itself supported by the technician includes the handles 22 and 23, the control device 10, the roll motor 9, and the bending motor 8, as shown in FIG. 12.

In addition, in the case where the position of the fixed point is not calculated in the image G, the field of view of the endoscope 2 is moved so that the fixed point is made to appear in the image G by bending the bending section 7 in this embodiment. Instead of this, the field of view may be widened by withdrawing the endoscope 2 in a direction along the longitudinal axis K of the insertion section 5 by means of the robot (advancement/withdrawal drive unit) 3 or the like, whereby the fixed point is made to appear in the image G. Because this eliminates the need for actuating the bending section 7, the position of the fixed point can be calculated by avoiding interference with tissue in the surroundings even in the case where the moving space of the bending section 7 at the distal end of the insertion section 5 is small.

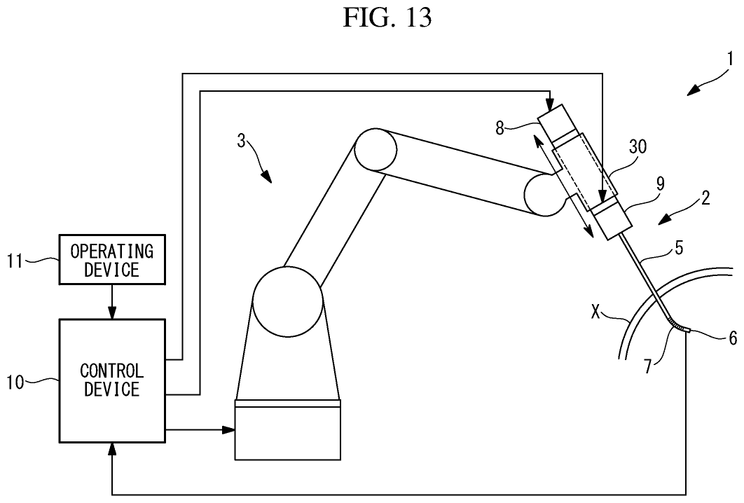

In this case, instead of the six-axis multi-joint robot that has been described as an example, a robot 3 including, at the distal end of a four-axis robot, a linear shaft (advancement/withdrawal drive unit) 30 for advancing/withdrawing the endoscope 2 in a direction along the longitudinal axis K of the insertion section 5 may be employed, as shown in FIG. 13. This allows the endoscope 2 to be advanced/withdrawn more easily in a direction along the longitudinal axis of the insertion section 5.

In addition, although this embodiment has been described by way of an example where the bending section 7 is bent in each of the four directions including the up, down, left, and right directions in order to make the center position P of the image G coincide with the fixed point, instead of this, the bending section 7 may be bent in a direction that intersects the calculated motion vectors and in which the magnitudes of the motion vectors become smaller by combining bending movements in the four directions.

In addition, although this embodiment has been described by way of example of the control device 10 as a unit that controls the endoscope 2 and performs image processing, instead of this, a unit that controls the endoscope 2 and a unit that performs image processing may be provided separately.

The above-described embodiment also leads to the following aspects.

One aspect of the present invention is an endoscope system including: an elongated section having a longitudinal axis; an image-capturing unit provided at a distal end of the elongated section; a bending section for changing a tilt angle of the image-capturing unit relative to the elongated section; a bending drive unit for driving the bending section; a rotation drive unit for rotating the elongated section about the longitudinal axis; and a control device for processing an image captured by the image-capturing unit, wherein the control device acquires, from the image-capturing unit, a plurality of images that are captured while the elongated section is being rotated by the rotation drive unit, calculates a position of a fixed point in an image on the basis of the plurality of acquired images, and determines a direction of the calculated position of the fixed point relative to a center of the image.

According to this aspect, the elongated section is inserted into the body, starting with the image-capturing unit, the elongated section is then rotated about the longitudinal axis by actuating the rotation drive unit, and a plurality of images that are captured by the image-capturing unit by actuating the image-capturing unit during the rotation are acquired. The control device calculates the position of a fixed point in an image on the basis of the plurality of acquired images and determines a direction of the position of the fixed point relative to the center of the image.

In other words, because rotation performed by the rotation drive unit causes the elongated section to rotate about the longitudinal axis, the images captured time-sequentially by the image-capturing unit during the rotation are images rotated about the fixed point, which is a point disposed on an extended line of the longitudinal axis. Therefore, it is possible to determine the direction of the position of the fixed point relative to the center of the image by driving the bending section by means of the bending drive unit, whereby the bending section can be straightened out with high accuracy.

In the above-described aspect, the control device may set a plurality of prospective fixed points on the basis of the plurality of images, calculate a motion vector of each of the prospective fixed points while the elongated section is being rotated, and calculate, as the position of the fixed point, a position of a prospective fixed point the calculated motion vector of which has the smallest magnitude.

With this configuration, in the case where a fixed point is present in the image, the position of the fixed point can be calculated with high accuracy. Therefore, it is possible to determine the direction of the highly accurately calculated position of the fixed point relative to the center of the image, whereby the bending section can be straightened out with high accuracy.

In addition, in the above-described aspect, the control device may calculate, as the position of the fixed point, the position of the prospective fixed point the motion vector of which has the smallest magnitude that is smaller than a predetermined threshold value.

With this configuration, in the case where no fixed points are present in the image, it is possible to prevent the prospective fixed point the motion vector of which has the smallest magnitude from being erroneously detected as a fixed point.

In addition, in the above-described aspect, in a case where the magnitude of the motion vector having the smallest magnitude is larger than the predetermined threshold value, the control device, by means of the bending drive unit, may cause the bending section to intersect the motion vector and move the bending section in a direction in which the magnitude of the motion vector becomes smaller.

With this configuration, the fixed point disposed at a position at which the magnitude of the motion vector is smallest can be brought into the image, so that the position of the fixed point can be calculated.

In addition, the above-described aspect may further include an advancement/withdrawal drive unit for advancing/withdrawing the elongated section in a direction along the longitudinal axis, wherein, in a case where the magnitude of the motion vector having the smallest magnitude is larger than the predetermined threshold value, the control device may cause the advancement/withdrawal drive unit to withdraw an endoscope backward in the direction along the longitudinal axis.

With this configuration, the field of view is widened by withdrawing the elongated section backward in the direction along the longitudinal axis, whereby the fixed point outside the image can be brought into the image.

In addition, in the above-described aspect, the control device may control the bending drive unit on the basis of the determined direction.

With this configuration, though the operation of the control device, it is possible to determine the direction of the fixed point, disposed at a position at which the magnitude of the motion vector is smallest, relative to the center of the image, whereby the bending section can be straightened out automatically with high accuracy.

In addition, in the above-described aspect, the control device may adjust an angle of the bending section in a direction in which a distance between the calculated position of the fixed point and the center becomes smaller.

With this configuration, it is possible to determine the direction of the fixed point, disposed at a position at which the magnitude of the motion vector is smallest, relative to the center of the image, whereby the bending section can be straightened out with high accuracy.

In addition, the above-described aspect may further include an information report unit for reporting the direction determined by the control device.

With this configuration, a technician can bend the bending section by operating the endoscope on the basis of reported information and determine the direction of the fixed point, disposed at a position at which the magnitude of the motion vector is smallest, relative to the center of the image, whereby the bending section can be manually straightened out with high accuracy.

In addition, another aspect of the present invention is a method for calibrating an endoscope that includes an elongated section having a longitudinal axis, an image-capturing unit provided at a distal end of the elongated section, and a bending section for changing a tilt angle of the image-capturing unit relative to the elongated section, the method including: acquiring a plurality of images that are captured by the image-capturing unit while the elongated section is being rotated; calculating a position of a fixed point in an image on the basis of the plurality of acquired images; determining a direction of the calculated position of the fixed point relative to a center of the image; and moving the bending section on the basis of the determined direction.

In the above-described aspect, the method may include: setting a plurality of prospective fixed points on the basis of the plurality of images; calculating a motion vector of each of the prospective fixed points while the elongated section is being rotated; and calculating, as the position of the fixed point, a position of a prospective fixed point the calculated motion vector of which has the smallest magnitude.

In addition, in the above-described aspect, the method may include: calculating, as the position of the fixed point, the position of the prospective fixed point the motion vector of which has the smallest magnitude that is smaller than a predetermined threshold value.

In addition, in the above-described aspect, the method may include: in a case where the magnitude of the motion vector having the smallest magnitude is larger than the predetermined threshold value, causing the bending section to intersect the motion vector, and moving the bending section in a direction in which the magnitude of the motion vector becomes smaller.

In addition, in the above-described aspect, the method may includes: in a case where the magnitude of the motion vector having the smallest magnitude is larger than the predetermined threshold value, withdrawing the endoscope backward in a longitudinal axis direction of the endoscope.

In addition, another aspect of the present invention is a control device of an endoscope, the control device including: at least one processor, wherein the processor is configured to: acquire, from an image-capturing unit of the endoscope, a plurality of images that are captured while an elongated section of the endoscope is being rotated by a rotation drive unit of the endoscope; calculate a position of a fixed point in an image on the basis of the plurality of acquired images; and determine a direction of the calculated position of the fixed point relative to a center of the image.

In the above-described aspect, the processor may be configured to: set a plurality of prospective fixed points on the basis of the plurality of images; calculate a motion vector of each of the prospective fixed points while the elongated section is being rotated; and calculate, as the position of the fixed point, a position of a prospective fixed point the calculated motion vector of which has the smallest magnitude.

In addition, in the above-described aspect, the processor may be configured to calculate, as the position of the fixed point, the position of the prospective fixed point the motion vector of which has the smallest magnitude that is smaller than a predetermined threshold value.

In addition, in the above-described aspect, in a case where the magnitude of the motion vector having the smallest magnitude is larger than the predetermined threshold value, the processor may be configured to cause a bending section of the endoscope to intersect the motion vector and move the bending section in a direction in which the magnitude of the motion vector becomes smaller.

In addition, in the above-described aspect, in a case where the magnitude of the motion vector having the smallest magnitude is larger than the predetermined threshold value, the processor may be configured to withdraw the endoscope backward in a longitudinal axis direction of the elongated section.

### REFERENCE SIGNS LIST

1, 21 Endoscope system
3 Robot (advancement/withdrawal drive unit)
5 Insertion section (elongated section)
6 Image-capturing unit
7 Bending section
8 Bending motor (bending drive unit)
9 Roll motor (rotation drive unit)
10 Control device
20 Information alarm unit
22 Handle (rotation drive unit)
23 Handle (bending drive unit)
30 Linear shaft (advancement/withdrawal drive unit)
G Image
K Longitudinal axis
P Center position (center)

1. An endoscope system comprising:
an elongated section having a longitudinal axis;
a bending section connected to a distal end of the elongated section, a tilt angle of the bending section relative to the longitudinal axis being changed;
an image-capturing unit provided at a distal end of the bending section;
a first motor that drives the bending section;
a second motor that rotates the elongated section about the longitudinal axis; and
a controller that processes an image captured by the image-capturing unit,
wherein the controller acquires, from the image-capturing unit, a plurality of images that are captured while the elongated section is being rotated by the second motor, and
calculates a driving direction of the bending section for making a center position of the image coincide with a position of a fixed point of the acquired plurality of images.

2. The endoscope system according to claim 1,
wherein the controller sets a plurality of feature points on the basis of the plurality of images,
calculates a motion vector of each of the feature points while the elongated section is being rotated, and
calculates, as the position of the fixed point, a position of a feature point the calculated motion vector of which has the smallest magnitude.

3. The endoscope system according to claim 1,
wherein the controller sets a plurality of feature points on the basis of the plurality of images,
calculates a motion vector of each of the feature points while the elongated section is being rotated, and
calculates, as the position of the fixed point, a position of a feature point the calculated motion vector of which has the smallest magnitude that is smaller than a predetermined threshold value.

4. The endoscope system according to claim 1, wherein the fixed point is a point disposed on an extension of the longitudinal axis.

5. The endoscope system according to claim 3,
wherein, in a case where the magnitude of the motion vector having the smallest magnitude is larger than the predetermined threshold value, the controller, by means of the first motor, causes the bending section to intersect the motion vector and moves the bending section in a direction in which the magnitude of the motion vector becomes smaller.

6. The endoscope system according to claim 3, further comprising:
a robot that advances/withdraws the elongated section in a direction along the longitudinal axis,
wherein, in a case where the magnitude of the motion vector having the smallest magnitude is larger than the predetermined threshold value, the controller causes the robot to withdraw an endoscope backward in the direction along the longitudinal axis.

7. The endoscope system according to claim 1,
wherein the controller controls the first motor on the basis of the calculated driving direction.

8. The endoscope system according to claim 7,
wherein the controller adjusts an angle of the bending section in a direction in which a distance between the position of the fixed point and the center becomes smaller.

9. The endoscope system according to claim 1, further comprising:
an information report unit configured to report the driving direction calculated by the controller.

10. A method for calibrating an endoscope that includes an image-capturing unit provided at a distal end of a benging section and an elongated section, the method comprising:
acquiring a plurality of images that are captured by the image-capturing unit while the elongated section is being rotated;
calculating a driving direction of the bending section for making a center position of an image coincide with a position of a fixed point of the acquired plurality of images; and
moving the bending section on the basis of the calculated driving direction.

11. The method for calibrating an endoscope according to claim 10, comprising:
setting a plurality of feature points on the basis of the plurality of images;
calculating a motion vector of each of the feature points while the elongated section is being rotated; and
calculating, as the position of the fixed point, a position of a feature point the calculated motion vector of which has the smallest magnitude.

12. The method for calibrating an endoscope according to claim 10, comprising:
setting a plurality of feature points on the basis of the plurality of images;
calculating a motion vector of each of the feature points while the elongated section is being rotated; and
calculating, as the position of the fixed point, a position of a feature point the calculated motion vector of which has the smallest magnitude that is smaller than a predetermined threshold value.

13. The method for calibrating an endoscope according to claim 10, wherein the fixed point is a point disposed on an extension of a longitudinal axis of the elongated section.

14. The method for calibrating an endoscope according to claim 12, comprising:
in a case where the magnitude of the motion vector having the smallest magnitude is larger than the predetermined threshold value, causing the bending section to intersect the motion vector, and moving the bending section in a direction in which the magnitude of the motion vector becomes smaller.

15. The method for calibrating an endoscope according to claim 12, comprising:
in a case where the magnitude of the motion vector having the smallest magnitude is larger than the predetermined threshold value, withdrawing the endoscope backward in a longitudinal axis direction of the endoscope.

16. A control device of an endoscope, the control device comprising:
at least one processor, wherein the processor is configured to:
acquire, from an image-capturing unit of the endoscope, a plurality of images that are captured while an elongated section of the endoscope is being rotated about a longitudinal axis of the elongated section;
calculate a driving direction of a bending section of the endoscope for making a center position of an image coincide with a position of a fixed point of the acquired plurality of images.

17. The control device of an endoscope, according to claim 16, wherein the processor is configured to:
set a plurality of feature points on the basis of the plurality of images;
calculate a motion vector of each of the feature points while the elongated section is being rotated; and
calculate, as the position of the fixed point, a position of a feature point the calculated motion vector of which has the smallest magnitude.

18. The control device of an endoscope, according to claim 16, wherein the processor is configured to:
set a plurality of feature points on the basis of the plurality of images;
calculate a motion vector of each of the feature points while the elongated section is being rotated; and
calculate, as the position of the fixed point, a position of a feature point the calculated motion vector of which has the smallest magnitude that is smaller than a predetermined threshold value.

19. The control device of an endoscope according to claim 18, wherein, in a case where the magnitude of the motion vector having the smallest magnitude is larger than the predetermined threshold value, the processor is configured to cause the bending section to intersect the motion vector and move the bending section in a direction in which the magnitude of the motion vector becomes smaller.

20. The control device of an endoscope according to claim 18, wherein, in a case where the magnitude of the motion vector having the smallest magnitude is larger than the predetermined threshold value, the processor is configured to withdraw the endoscope backward in a longitudinal axis direction of the elongated section.
